(19) United States
(12) Patent Application Publication
(10) Pub. No.: US 2025/0234924 A1
(43) Pub. Date: Jul. 24, 2025

(54) LIQUID GUIDING ASSEMBLY, HEATING ASSEMBLY, ATOMIZER, AND ELECTRONIC ATOMIZATION DEVICE

(71) Applicant: SHENZHEN SMOORE TECHNOLOGY LIMITED, Shenzhen (CN)

(72) Inventors: Yaqiao WANG, Shenzhen (CN); Run CAO, Shenzhen (CN); Shouhao CHEN, Shenzhen (CN); Hui DAI, Shenzhen (CN)

(21) Appl. No.: 19/176,826

(22) Filed: Apr. 11, 2025

Related U.S. Application Data
(63) Continuation of application No. PCT/CN2023/080463, filed on Mar. 9, 2023.

(30) Foreign Application Priority Data
Oct. 12, 2022 (CN) 202211249457.7

(51) Int. Cl. A24F 40/44; A24F 40/10; A24F 40/46; A24F 40/485; A61M 11/04; B32B 5/02; B32B 15/14; B32B 15/20

(52) U.S. Cl. CPC A24F 40/44; A24F 40/10; A24F 40/46; A24F 40/485; A61M 11/042; B32B 5/028; B32B 15/14; B32B 15/20; A61M 2205/3633; B32B 2262/062; B32B 2307/302

(57) ABSTRACT

A liquid guiding assembly for an electronic atomization device includes: at least one thermal conduction layer and at least one isolation layer that are stacked. The at least one isolation layer makes contact with a heating element of the electronic atomization device, the at least one isolation layer isolating the thermal conduction layer from the heating element. The at least one isolation layer transfers at least some heat generated by the heating element to the at least one thermal conduction layer.

### CROSS-REFERENCE TO PRIOR APPLICATION

This application is a continuation of International Patent Application No. PCT/CN2023/080463, filed on Mar. 9, 2023, which claims priority to Chinese Patent Application No. 202211249457.7, filed on Oct. 12, 2022. The entire disclosure of both applications is hereby incorporated by reference herein.

### FIELD

This application relates to the field of electronic atomization technologies, and in particular, to a liquid guiding assembly, a heating assembly, an atomizer, and an electronic atomization device.

### BACKGROUND

An electronic atomization device is an electronic delivery system that controls an operating state and vapor output through a control circuit and atomization elements, and produces aerosols of different compositions for people to inhale according to different aerosol-forming materials.

In the related art, when the viscosity of the aerosol-forming materials is high, the fluidity of the aerosol-forming materials is poor, which can lead to liquid guiding obstruction in a liquid guiding assembly. Meanwhile, the high-viscosity aerosol-forming materials may impede the flow of ventilation bubbles, which is unfavorable for elimination of the ventilation bubbles, resulting in the formation of bubbles of various sizes. When the ventilation bubbles are excessive or too large, the problem of blockage of the ventilation bubbles is likely to be caused, leading to liquid absorption obstruction in a heating assembly. Poor liquid absorption can lead to dry heating, affecting the service life of the electronic atomization device and use experience of users.

### SUMMARY

In an embodiment, the present invention provides a liquid guiding assembly for an electronic atomization device, including: at least one thermal conduction layer and at least one isolation layer that are stacked, wherein the at least one isolation layer makes contact with a heating element of the electronic atomization device, the at least one isolation layer isolating the thermal conduction layer from the heating element, and wherein the at least one isolation layer is configured to transfer at least some heat generated by the heating element to the at least one thermal conduction layer.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a cross-sectional view of an atomizer according to an embodiment of this application;

FIG. 2 is a cross-sectional view of a liquid guiding assembly arranged on a support element shown in FIG. 1;

FIG. 3 is a schematic structural diagram of a heating assembly according to an embodiment of this application;

FIG. 4 is a schematic structural diagram of a support element according to an embodiment of this application;

FIG. 5 is a schematic structural diagram of the support element from another perspective shown in FIG. 4; and

FIG. 6 is a schematic structural diagram the support element from yet another perspective shown in FIG. 4.

### DETAILED DESCRIPTION

In an embodiment, the present invention provides a liquid guiding assembly, a heating assembly, and an electronic atomization device, which are good in liquid guiding effect.

In an embodiment, the present invention provides a liquid guiding assembly, used in an electronic atomization device. The liquid guiding assembly includes a thermal conduction layer and an isolation layer which are stacked, the isolation layer makes contact with a heating element of the electronic atomization device, and the isolation layer is used for isolating the thermal conduction layer from the heating element; and

the isolation layer can transfer some of heat generated by the heating element to the thermal conduction layer.

In an embodiment, the isolation layer is sleeved over a periphery of the thermal conduction layer. In an embodiment, the thermal conduction layer is a metal mesh layer.

In an embodiment, the thermal conduction layer is a copper alloy. In an embodiment, the isolation layer is a cotton layer.

In an embodiment, the porosity of the thermal conduction layer ranges from 0.45 to 0.99.

In an embodiment, the permeability of the thermal conduction layer ranges from 1×10⁻¹¹ m² to 1×10⁻⁹ m².

In an embodiment, the porosity of the isolation layer ranges from 0.45 to 0.99.

In an embodiment, the permeability of the isolation layer ranges from 1×10⁻¹¹ m² to 1×10⁻⁹ m².

In an embodiment, a plurality of thermal conduction layers and a plurality of isolation layers are provided, and are alternately arranged.

An embodiment of this application provides a heating assembly, including a heating element and the above liquid guiding assembly, and the heating element is arranged on the isolation layer.

In an embodiment, the heating assembly includes a support element, the support element includes a body, a liquid guiding channel, and liquid guiding holes, the liquid guiding channel penetrates through the two ends of the body along an axial direction of the body, and the liquid guiding holes penetrate through the side wall of the liquid guiding channel along a radial direction of the body; and

the thermal conduction layer is sleeved over a periphery of the support element, the isolation layer is sleeved over a periphery of the thermal conduction layer, and an aerosol-forming material within the liquid guiding channel can be guided to the liquid guiding assembly via the liquid guiding holes.

In an embodiment, the support element is a metal element.

In an embodiment, the length of the body along the axial direction is greater than the length of the liquid guiding assembly along the axial direction of the body.

In an embodiment, the length of the thermal conduction layer along the axial direction is greater than the length of the isolation layer along the axial direction.

In an embodiment, the equivalent hole diameter of the liquid guiding holes ranges from 0.01 mm to 3 mm.

In an embodiment, the inner diameter of the body ranges from 0.3 mm to 3 mm.

In an embodiment, the axial length of the body ranges from 3 mm to 30 mm.

In an embodiment, the wall thickness of the body ranges from 0.05 mm to 0.2 mm.

An embodiment of this application provides an atomizer, including a liquid storage cavity and the above heating assembly. The liquid storage cavity is used for storing an aerosol-forming material, and the aerosol-forming material within the liquid storage cavity can be guided to the heating element via the liquid guiding assembly.

An embodiment of this application provides an electronic atomization device, including a power supply assembly and the above atomizer. The power supply assembly is electrically connected with the heating assembly.

The liquid guiding assembly provided in this embodiment of this application includes the thermal conduction layer and the isolation layer which are stacked. The isolation layer makes contact with the heating element of the electronic atomization device. The isolation layer is used for isolating the thermal conduction layer from the heating element. The thermal conduction layer may guide the aerosol-forming material of the electronic atomization device to the isolation layer. In other words, part of the aerosol-forming material of the electronic atomization device may be guided to the heating element sequentially via the thermal conduction layer and the isolation layer, and the heating element may heat and atomize the aerosol-forming material to generate aerosols. Additionally, by arranging the thermal conduction layer, the isolation layer may transfer part of the heat generated by the heating element to the thermal conduction layer. The thermal conduction layer may conduct the heat to the nearby aerosol-forming material more quickly. After the aerosol-forming material near the thermal conduction layer heats up, the viscosity is reduced, thereby improving the fluidity of the aerosol-forming material near the thermal conduction layer. Therefore, on one hand, elimination of ventilation bubbles is facilitated, and a ventilation channel can be prevented from being blocked while ventilation is improved. On the other hand, the improved fluidity of the aerosol-forming material near the thermal conduction layer facilitates the guidance of the aerosol-forming material to the heating element via the liquid guiding assembly, thereby improving the liquid guiding effect of the liquid guiding assembly.

### DESCRIPTIONS OF REFERENCE NUMERALS

1000—atomizer; 1000a—liquid storage cavity; 100—heating assembly; 10—support element; 10a—liquid guiding channel; 10b—liquid guiding hole; 11—body; 20—liquid guiding assembly; 21—thermal conduction layer; 22—isolation layer; 30—heating element; 200—housing; and 200a—air outlet channel.

It should be noted that embodiments and technical features in the embodiments in this application may be combined without conflicts. Detailed descriptions in specific embodiments should be understood as explanatory notes for the purpose of this application and should not be regarded as improper limitations on this application.

An embodiment of this application provides an electronic atomization device, including an atomizer provided in any one of embodiments of this application.

It should be noted that a specific type of the electronic atomization device is not limited here. Exemplarily, in some embodiments, the electronic atomization device may be an e-cigarette, a medical electronic atomization device, a cosmetic electronic atomization device, or the like.

The electronic atomization device is used for atomizing an aerosol-forming material to generate aerosols for user inhalation. The aerosol-forming material includes, but is not limited to, medications, nicotine-containing materials, nicotine-free materials, or the like.

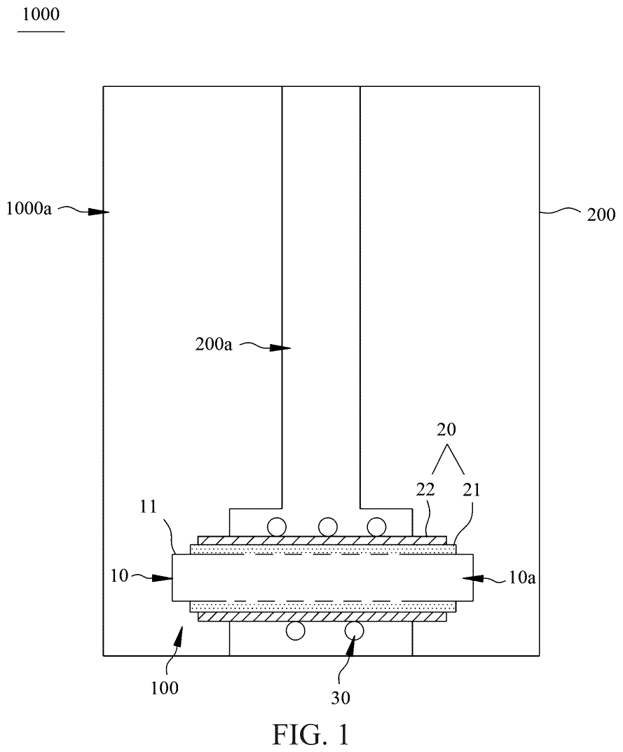

An embodiment of this application provides an atomizer. Referring to FIG. 1, the atomizer 1000 includes a liquid storage cavity 1000a and a heating assembly provided in any one of the embodiments of this application. The liquid storage cavity 1000a is used for storing an aerosol-forming material, and the aerosol-forming material in the liquid storage cavity 1000a may be guided to a heating element 30 by a liquid guiding assembly 20 of the heating assembly 100.

Exemplarily, the electronic atomization device includes a main unit. The main unit includes a power supply assembly. The power supply assembly may include, for example, a battery. The power supply assembly is electrically connected with the heating assembly 100 of the atomizer 1000. In other words, the power supply assembly is used for supplying power to the heating assembly 100, thereby allowing the heating assembly 100 to heat and atomize the aerosol-forming material.

It should be noted that the atomizer 1000 and the main unit may be of an integrally-formed structure or a split structure. For example, the atomizer 1000 may be detachably connected with the main unit.

In an embodiment, please continue to refer to FIG. 1. The atomizer 1000 includes a housing 200. The housing 200 forms an air outlet channel 200a. The aerosols generated by the aerosol-forming material are inhaled by a user through the air outlet channel 200a. It should be noted that a specific method for using the electronic atomization device is not limited here. For example, the user may inhale the aerosols through the housing 200 or through an additional suction nozzle in conjunction with the housing 200.

An embodiment of this application provides a heating assembly. Referring to FIG. 1 to FIG. 3, the heating assembly 100 includes the heating element 30 and a liquid guiding assembly 20 provided in any one of the embodiments of this application, and the heating element 30 is arranged on an isolation layer 22.

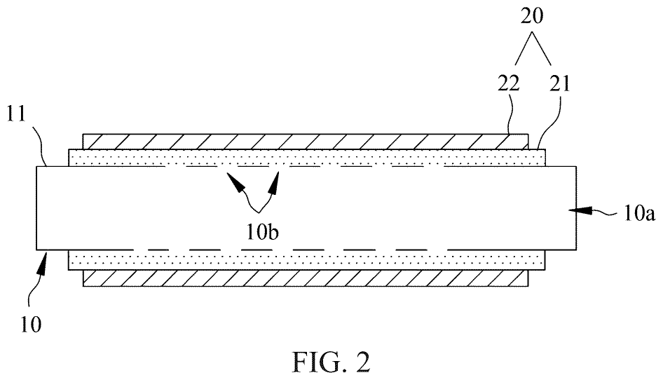

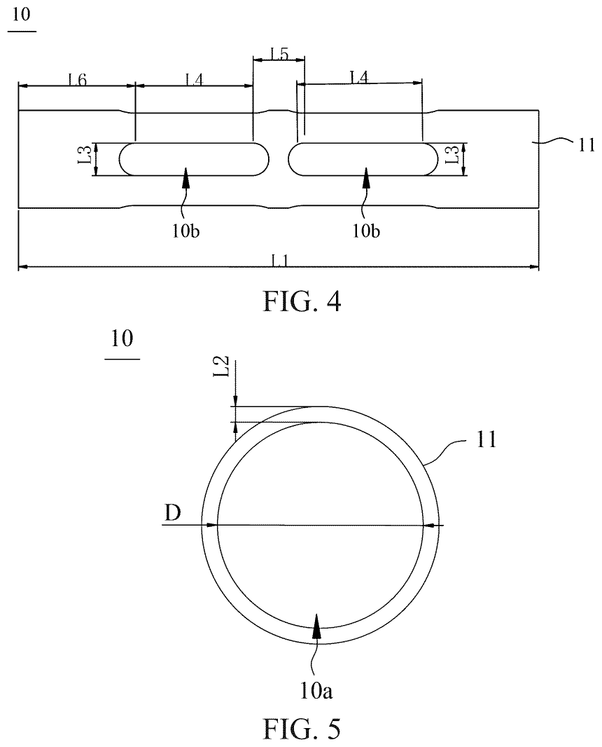

Referring to FIG. 2, and FIG. 4 to FIG. 6, the heating assembly 100 includes a support element 10. The support element 10 includes a body 11, a liquid guiding channel 10a, and liquid guiding holes 10b. The liquid guiding channel 10a penetrates through the two ends of the body 11 along an axial direction of the body 11, and the liquid guiding holes 10b penetrate through the side wall of the liquid guiding channel 10a along a radial direction of the body 11. The aerosol-forming material within the liquid storage cavity 1000a may enter the liquid guiding channel 10a via at least one end of the body 11. The liquid guiding assembly 20 is sleeved over a periphery of the support element 10 and covers the outer sides of the liquid guiding holes 10b. The aerosol-forming material within the liquid guiding channel 10a may be guided to the liquid guiding assembly 20 via the liquid guiding holes 10b. The heating element 30 is arranged on the liquid guiding assembly 20 for heating and atomizing the aerosol-forming material, to generate the aerosols for user inhalation.

Certainly, the aerosol-forming material within the liquid storage cavity 1000a may also be guided to the liquid guiding assembly 20 without passing through the liquid guiding channel 10a or the liquid guiding holes 10b. For example, liquid absorption may be directly achieved through a part of the liquid guiding assembly 20 that extends into the liquid storage cavity 1000a or communicates with the liquid storage cavity 1000a.

At least one end of the liquid guiding channel 10a of the heating assembly 100 communicates with the liquid storage cavity 1000a, thereby allowing the heating assembly 100 to heat and atomize the aerosol-forming material to form the aerosols for user inhalation.

It should be noted that at least one end of the liquid guiding channel 10a communicating with the liquid storage cavity 1000a means that one end of the liquid guiding channel 10 may communicate with the liquid storage cavity 1000a, or both the two ends may communicate with the liquid storage cavity 1000a. In this embodiment of this application, the description is made with the example of the two ends of the liquid guiding channel 10a communicating with the liquid storage cavity 1000a, and therefore the amount of the aerosol-forming material entering the heating assembly 100 is increased, and meanwhile the aerosol-forming material is evenly guided to the liquid guiding assembly 20 via the liquid guiding channel 10a, thereby improving an atomization effect.

An embodiment of this application provides a liquid guiding assembly. Referring to FIG. 1 and FIG. 2, the liquid guiding assembly 20 includes a thermal conduction layer 21 and an isolation layer 22 which are stacked. The isolation layer 22 makes contact with the heating element 30 of the electronic atomization device. The isolation layer 22 is used for isolating the thermal conduction layer 21 from the heating element 30. The thermal conduction layer 21 may guide the aerosol-forming material of the electronic atomization device to the isolation layer 22. In other words, part of the aerosol-forming material of the electronic atomization device may be guided to the heating element 30 sequentially via the thermal conduction layer 21 and the isolation layer 22, and the heating element 30 may heat and atomize the aerosol-forming material to generate the aerosols.

It should be noted that the thermal conduction layer 21 has a high thermal conductivity. When the heating element 30 heats up during operation, the isolation layer 22 may transfer some of heat generated by the heating element 30 to the thermal conduction layer 21. The thermal conduction layer 21 can quickly absorb heat and then quickly transfer the heat to the aerosol-forming material near the thermal conduction layer 21, thereby reducing the viscosity of the aerosol-forming material near the thermal conduction layer 21, and then improving fluidity of the aerosol-forming material near the thermal conduction layer 21.

The isolation layer 22 makes contact with the heating element 30. The isolation layer 22 is used for isolating the thermal conduction layer 21 from the heating element 30. In other words, the aerosol-forming material is guided to the heating element 30 sequentially via the thermal conduction layer 21 and the isolation layer 22, thereby effectively preventing excessive heat loss in a temperature rise phase, and then reducing energy consumption and shortening preheating time.

A specific method for stacking the thermal conduction layer 21 and the isolation layer 22 is not limited here. Exemplarily, in an embodiment, referring to FIG. 1 and FIG. 2, the isolation layer 22 is sleeved over a periphery of the thermal conduction layer 21. In other words, the isolation layer 22 and the thermal conduction layer 21 are in a ring shape, and the isolation layer 22 is sleeved over the periphery of the thermal conduction layer 21 to form a composite liquid guiding layer.

The thermal conduction layer 21 is wound around the support element 10, and the isolation layer 22 is wound around the thermal conduction layer 21 to form the composite liquid guiding layer. The heating element 30 is wound around the isolation layer 22, thereby allowing the isolation layer 22 to isolate the thermal conduction layer 21 from the heating element 30. In some other embodiments, the thermal conduction layer 21 and the isolation layer 22 may be stacked together to form the composite liquid guiding layer. In yet other embodiments, the thermal conduction layer 21 and the isolation layer 22 may be woven together to form the composite liquid guiding layer. For example, metal and cotton are woven together to form the composite liquid guiding layer.

It should be noted that the support element 10 exemplified in this embodiment of this application may be a tubular member for containing the aerosol-forming material. The tubular member is similar to a cylindrical shape, but is not used for limiting the shape of the support element 10 in this embodiment of this application to be similar to the cylindrical shape. The support element 10 in this embodiment of this application may be in a shape similar to a triangular prism, an elliptical cylinder, or other shapes.

It should be noted that the number of thermal conduction layers 21 is not limited here. For example, there may be one or more thermal conduction layers 21, and the number of the thermal conduction layers 21 is determined according to specific conditions.

It should be noted that the number of isolation layers 22 is not limited here. For example, there may be one or more isolation layers 22, and the number of the isolation layers 22 is determined according to specific conditions.

It should be noted that in this embodiment of this application, multilayer refers to two or more layers.

In some embodiments, a plurality of thermal conduction layers 21 and a plurality of isolation layers 22 are provided. The thermal conduction layers 21 and the isolation layers 22 are alternately arranged. Accordingly, a liquid storage capacity of the liquid guiding assembly 20 is improved, and meanwhile the thermal conduction layers 21 can transfer some of the heat to the nearby aerosol-forming material more quickly.

Materials of the thermal conduction layers 21 may be consistent, or inconsistent materials may also be selected according to actual conditions. Materials of the isolation layers 22 may be consistent, or inconsistent materials may also be selected according to actual conditions.

In some other embodiments, the thermal conduction layer 21 may be a single layer while a plurality of isolation layers 22 may be provided.

In yet other embodiments, a plurality of thermal conduction layers 21 may be provided while the isolation layer 22 may be a single layer.

The high-viscosity aerosol-forming material has a working characteristic that the viscosity is reduced as the temperature increases. For example, at room temperature, the viscosity reaches up to 106 cP (centipoise), but when the temperature rises to around 70° C., the viscosity drops to 1000 cP or below. In the related art, when the viscosity of the aerosol-forming material is high, the fluidity of the aerosol-forming material is poor, leading to liquid guiding obstruction of the liquid guiding assembly 20. Poor liquid absorption may result in dry heating, affecting the service life of the electronic atomization device and use experience of the user.

The liquid guiding assembly provided in this embodiment of this application includes the thermal conduction layer 21 and the isolation layer 22 which are stacked. The isolation layer 22 makes contact with the heating element 30 of the electronic atomization device. The isolation layer 22 is used for isolating the thermal conduction layer 21 from the heating element 30. The thermal conduction layer 21 may guide the aerosol-forming material of the electronic atomization device to the isolation layer 22. In other words, part of the aerosol-forming material of the electronic atomization device may be guided to the heating element 30 sequentially via the thermal conduction layer 21 and the isolation layer 22, and the heating element 30 may heat and atomize the aerosol-forming material to generate the aerosols. Additionally, by arranging the thermal conduction layer 21, the isolation layer 22 may transfer part of the heat generated by the heating element 30 to the thermal conduction layer 21. The thermal conduction layer 21 may conduct the heat to the nearby aerosol-forming material more quickly. After the aerosol-forming material near the thermal conduction layer 21 heats up, the viscosity is reduced, thereby improving the fluidity of the aerosol-forming material near the thermal conduction layer 21. Therefore, on one hand, elimination of the ventilation bubbles is facilitated, and a ventilation channel can be prevented from being blocked while ventilation is improved. On the other hand, the improved fluidity of the aerosol-forming material near the thermal conduction layer 21 facilitates the guidance of the aerosol-forming material to the heating element 30 via the liquid guiding assembly 20, thereby improving the liquid guiding effect of the liquid guiding assembly 20.

It should be noted that a specific material of the thermal conduction layer 21 is not limited. Exemplarily, in an embodiment, the thermal conduction layer 21 is a metal mesh layer, and in other words, the thermal conduction layer 21 is made of a metal material. Accordingly, controlling the size of the thermal conduction layer 21 and forming a mesh structure are facilitated during manufacturing. In a machining process of the metal material, size precision and errors can be well controlled, thereby achieving higher processing precision, such as being made thinner. Meanwhile, the metal material has good thermal conduction performance. That is, the metal mesh layer can be quickly heated, thereby quickly transferring the heat to the aerosol-forming material near the thermal conduction layer 21. Additionally, the metal mesh layer may also store a part of aerosol-forming material with the reduced viscosity due to porous performance, thereby continuously and quickly supplying the aerosol-forming material with the reduced viscosity to the isolation layer 22, and heating and atomizing the aerosol-forming material on the isolation layer 22.

Exemplarily, the thermal conduction layer 21 is a copper alloy. A material of the metal mesh layer may be selected from metals or alloys with different thermal conduction performance according to characteristics of the aerosol-forming material, such as stainless steel, nickel, aluminum, brass, red copper, and other metallic elements or alloys. That is, the above materials are used for being woven into the metal mesh layer.

Certainly, the thermal conduction layer 21 may also be made of non-metallic materials with a good thermal conductivity coefficient.

In an embodiment, the isolation layer 22 is a cotton layer, and a specific material of the cotton layer is not limited. Exemplarily, the material of the cotton layer may be natural organic cotton or organic synthetic polymeric porous foam cotton. The cotton layer, made of a cotton fiber material, can stably store part of aerosol-forming material and quickly guide the aerosol-forming material from one side of the thermal conduction layer 21 to the heating element 30. The heating element 30 in a power-on state heats the aerosol-forming material on the cotton layer to form the aerosols. Additionally, when the thermal conduction layer 21 is the metal mesh layer, the isolation layer 22 may also isolate the metal mesh layer from the heating element 30 through the cotton layer, thereby avoiding an electrical connection between the heating element 30 and the liquid guiding assembly 20, and then improving safety performance of the atomizer 1000.

In an embodiment, the porosity of the thermal conduction layer 21 ranges from 0.45 to 0.99. When the porosity of the thermal conduction layer 21 is lower than 0.45, the liquid supply amount may be influenced, and the amount of vapor is reduced. When the porosity of the thermal conduction layer 21 is higher than 0.99, structural strength of the thermal conduction layer 21 may be influenced.

In an embodiment, the permeability of the thermal conduction layer 21 ranges from 1×10⁻¹¹ m² to 1×10⁻⁹ m². The permeability refers to the ability to allow fluid passage under a certain pressure difference and is a parameter characterizing the liquid conduction capacity of the thermal conduction layer 21. When the permeability of the thermal conduction layer 21 is lower than 1×10¹¹ m², the liquid supply amount may be influenced, and the amount of vapor is reduced. When the permeability of the thermal conduction layer 21 is higher than 1×10⁻⁹ m², liquid leakage may be caused.

In an embodiment, the porosity of the isolation layer 22 ranges from 0.45 to 0.99. When the porosity of the isolation layer 22 is lower than 0.45, the liquid supply amount may be influenced, and the amount of vapor is reduced. When the porosity of the isolation layer 22 is higher than 0.99, structural strength of the isolation layer 22 may be influenced.

In an embodiment, the permeability of the isolation layer 22 ranges from 1×10⁻¹¹ m² to 1×10⁻⁹ m². The permeability refers to the ability to allow fluid passage under a certain pressure difference and is a parameter characterizing the liquid conduction capacity of the isolation layer 22. When the permeability of the isolation layer 22 is lower than 1×10⁻¹¹ m², the liquid supply amount may be influenced, and the amount of vapor is reduced. When the permeability of the isolation layer 22 is higher than 1×10⁻⁹ m², liquid leakage may be caused.

In an embodiment, referring to FIG. 1 to FIG. 3, the heating assembly 100 includes the support element 10, a liquid guiding element, and the heating element 30. In other words, the aerosol-forming material within the liquid storage cavity 1000a may enter the liquid guiding channel 10a via at least one end of the body 11 of the heating assembly 100. The thermal conduction layer 21 is sleeved over the periphery of the support element 10. The isolation layer 22 is sleeved over the periphery of the thermal conduction layer 21. The liquid guiding assembly 20 covers the outer sides of the liquid guiding holes 10b. The aerosol-forming material within the liquid guiding channel 10a may be guided to the liquid guiding assembly 20 via the liquid guiding holes 10b. The heating element 30 is arranged on the isolation layer 22, and is used for heating and atomizing the aerosol-forming material on the isolation layer 22 to generate the aerosols for user inhalation.

Exemplarily, referring to FIG. 1 to FIG. 6, the support element 10 is a hollow tube with two through ends. A tube cavity of the hollow tube forms the liquid guiding channel 10a, and the liquid guiding holes 10b penetrate through the side wall of the hollow tube along a radial direction of the hollow tube. The thermal conduction layer 21 is wound around the side wall of the hollow tube, and the isolation layer 22 is wound around the thermal conduction layer 21 to form a composite liquid guiding layer. The heating element 30 is wound around the isolation layer 22.

It should be noted that the specific shape of the liquid guiding holes 10b is not limited here, and the liquid guiding holes include, but are not limited to, circular holes, elliptical holes, elongated holes, square holes, etc.

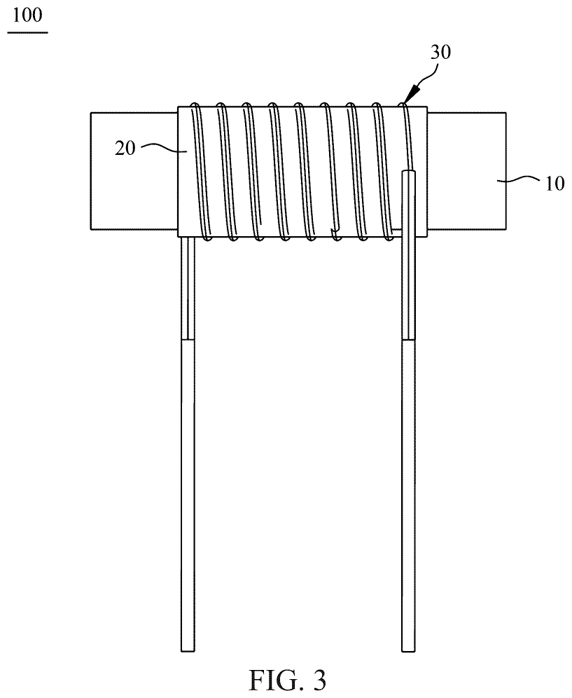

Exemplarily, referring to FIG. 3 to FIG. 6, the liquid guiding holes 10b include at least two rows of elongated holes distributed along the axial direction of the body 11. Accordingly, the plurality of rows of elongated holes not only facilitate the guidance of a larger number of aerosol-forming material from the liquid guiding channel 10a into the liquid guiding assembly 20 via the elongated holes, but also enhance the liquid guiding capacity of the liquid guiding assembly 20 and the atomization efficiency of the heating assembly 100 by increasing the liquid supply area of the support element 10. Additionally, a situation that blockage of any elongated hole leads to unavailable guidance of the aerosol-forming material from the liquid guiding channel 10a into the liquid guiding assembly 20 can be avoided. Exemplarily, as shown in FIG. 3 to FIG. 6, the support element 10 has two rows of elongated holes distributed along the axial direction of the body 11, which may ensure both sufficient length of the elongated holes and the liquid supply area of the support element 10. If there are three or more rows of the elongated holes distributed along the axial direction of the body 11, the elongated holes may be too short, thereby reducing the liquid supply area of the support element 10. If only one row is provided and the liquid guiding holes 10 b are too short, the liquid guiding area is insufficient, and if the liquid guiding holes 10b are too long, strength is insufficient.

It should be noted that in this embodiment of this application, “at least two rows” refers to two or more rows.

It should be noted that the elongated holes may be rectangular holes. In this embodiment of this application, the two ends of each elongated hole are provided with arc smooth transitions, which not only enhances the strength of the body 11 but also makes the body 11 more attractive. In a specific embodiment, referring to FIG. 4, the diameter of arcs at the two ends of each elongated hole is equal to the width of the elongated hole.

Referring to FIG. 1 and FIG. 2, the heating element 30 is arranged on the isolation layer 22 of the liquid guiding assembly 20, for heating and atomizing the aerosol-forming material to generate the aerosols for user inhalation. The heating element 30 includes, but is not limited to, a heating wire, a heating mesh, and a heating strip, where the heating wire may be, for example, a circular heating wire, which certainly, may also be a heating wire in another shape. In this embodiment of this application, the heating element 30 is exemplified as a heating wire spirally wound around the periphery of the isolation layer 22.

It should be noted that the specific shape of the liquid guiding channel 10a is not limited here. The shape of the cross section of the liquid guiding channel 10a includes, but is not limited to, a circle, an oval, or a polygon with rounded corners, such as a triangle with rounded corners. Exemplarily, in an embodiment, referring to FIG. 1, FIG. 2, and FIG. 5, the shape of the cross section of the liquid guiding channel 10a is the circle, which facilitates smooth flow of the aerosol-forming material within the liquid guiding channel 10a. The shape of the cross section of the liquid guiding channel 10a refers to the shape of the section of the liquid guiding channel 10a taken along a plane perpendicular to the axial direction of the body 11.

It should be noted that the liquid guiding holes 10b of the support element 10 are typically formed by laser processing. Compared to the elongated holes, processing an array of circular holes with the diameters of hundreds of micrometers on a circumference requires a longer laser travel path and takes more time, resulting in lower processing efficiency and increased processing costs.

The heating assembly provided in this embodiment of this application includes the support element 10, the liquid guiding assembly 20, and the heating element 30. The support element 10 includes the body 11, the liquid guiding channel 10a, and the liquid guiding holes 10b. The liquid guiding channel 10a penetrates through the two ends of the body 11 along the axial direction of the body 11, and the liquid guiding holes 10b penetrate through the side wall of the liquid guiding channel 10a along the radial direction of the body 11. It should be understood that the liquid supply area of the support element 10 refers to the total area of openings of all the liquid guiding holes 10b in the body 11. By configuring the liquid guiding holes 10b to include at least two rows of elongated holes distributed along the axial direction of the body 11, on one hand, the liquid supply area of the support element 10 is increased, a more aerosol-forming material for atomization exists on the support element 10, the liquid supply capacity and the atomization efficiency of the heating assembly 100 are improved, and the aerosol-forming amount of the heating assembly 100 within unit time is larger when the temperature is kept unchanged. On the other hand, at least two rows of elongated holes are distributed along the axial direction of the body 11, the two adjacent rows of elongated holes may be connected through the side wall of the liquid guiding channel 10a on the premise of ensuring that the support element 10 has a certain liquid supply area, thereby improving structural strength of the support element 10. Additionally, by configuring the liquid guiding holes 10b to include the at least two rows of elongated holes distributed along the axial direction of the body 11, compared to circular holes or other holes, under the same liquid supply area, the cutting perimeter required for processing the elongated holes is short, processing is easy, and processing steps are reduced, thereby improving the processing efficiency and reducing processing costs.

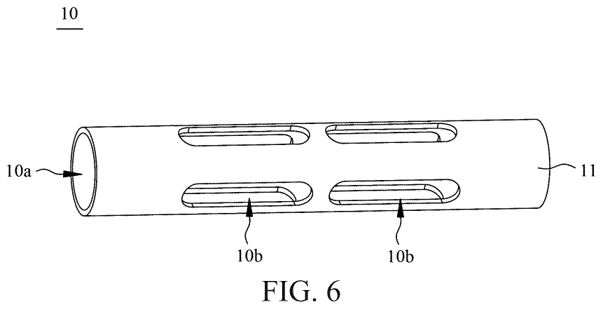

In an embodiment, referring to FIG. 4 and FIG. 6, the elongated holes extend along the axial direction of the body 11. In other words, the length direction of the elongated holes is arranged along the axial direction of the body 11, meaning that the length direction of the elongated holes is the same as the axial direction of the body 11. Therefore, the two adjacent rows of elongated holes may be connected through the side wall of the liquid guiding channel 10a on the premise of ensuring that the support element 10 has a certain liquid supply area, thereby enhancing the structural strength of the support element 10. In addition, the processing efficiency is also improved.

In an embodiment, the elongated holes extend along the circumferential direction of the body 11. In other words, the length direction of the elongated holes is arranged along the circumferential direction of the body 11, meaning that the length direction of the elongated holes is approximately perpendicular to the axial direction of the body 11. Therefore, the two adjacent rows of elongated holes may be connected through the side wall of the liquid guiding channel 10a on the premise of ensuring that the support element 10 has a certain liquid supply area, thereby enhancing the structural strength of the support element 10. More elongated holes may also be arranged along the axial direction of the body 11, thereby further increasing the liquid supply area of the support element 10. In addition, the processing efficiency is also improved.

In some other embodiments, the extension direction of the elongated holes is inclined relative to the axial direction of the body 11, meaning that the length direction of the elongated holes is set at a certain angle relative to the axial direction of the body 11.

In an embodiment, the support element 10 is a metal element. In other words, the support element 10 is made of a metal material. Accordingly, the size of the support element 10 is conveniently controlled in the manufacturing process. In the processing process, size precision and errors of the metal material can be well controlled, thereby achieving higher processing precision, such as being made thinner. Meanwhile, the metal material has certain thermal conduction performance, thereby improving the atomization efficiency of the heating assembly 100. That is, the support element 10 made of the metal material has the functions of support, liquid guiding, and thermal conduction at the same time. By improving the thermal conduction performance of the support element 10, the thermal conduction layer 21 may conduct the heat to the support element 10 more quickly, and then the support element 10 conducts the heat to the aerosol-forming material nearby, within the liquid guiding channel 10a, or within the liquid storage cavity 1000a more quickly. After the aerosol-forming material heats up, the viscosity is reduced, thereby improving the fluidity of the aerosol-forming material nearby the support element 10, within the liquid guiding channel 10a, or within the liquid storage cavity 1000a. Ventilation is improved, and meanwhile the aerosol-forming material is facilitated to be guided to the heating element 30 via the liquid guiding assembly 20, thereby improving the liquid guiding effect of the liquid guiding assembly 20.

Exemplarily, the support element 10 may be, for example, made of stainless steel, an aluminum alloy, and a brass alloy. In this embodiment of this application, the support element 10 may be, for example, made of 304 stainless steel.

Certainly, the support element 10 may also be made of glass. The glass is specifically any one of borosilicate glass, quartz glass, or photosensitive lithium aluminosilicate glass. In some other embodiments, the support element 10 may also be made of ceramic, metal, rigid plastic, polymer, or other inorganic non-metallic materials, and is a component with certain mechanical strength.

The support element 10 is made of the metal material. Under the condition of ensuring strength and safety, by using the metal material, the support element 10 may be properly thinned, that is, the wall thickness of the body 11 may be properly reduced, thereby reducing the mass of the support element 10, reducing thermal capacity consumption of the support element 10, and improving the heating efficiency of the heating assembly 100. Accordingly, with the same outer diameter, the inner diameter of the body 11 may be larger, and macroscopic flow resistance in the liquid guiding channel 10a is smaller. Additionally, by reducing the wall thickness of the body 11, a path for the aerosol-forming material to flow from the liquid guiding channel 10a to the liquid guiding assembly 20 is shortened, thereby further reducing the flow resistance of the aerosol-forming material, and then improving the liquid supply capacity and the atomization effect of the heating assembly 100.

In an embodiment, the liquid guiding holes 10b include at least two columns of elongated holes distributed along the circumferential direction of the body 11. Exemplarily, referring to FIG. 6, four columns of elongated holes are distributed along the circumferential direction of the body 11. Accordingly, the plurality of columns of elongated holes not only facilitate the guidance of a larger amount of aerosol-forming material from the liquid guiding channel 10a into the liquid guiding assembly 20 via the elongated holes, but also enhance the liquid guiding capacity and the atomization efficiency of the heating assembly 100 by increasing the liquid supply area of the support element 10. Additionally, a situation that blockage of any column of elongated holes leads to unavailable guidance of the aerosol-forming material from the liquid guiding channel 10a into the liquid guiding assembly 20 can be avoided.

It should be noted that in this embodiment of this application, “at least two columns” refers to two or more columns.

In an embodiment, two adjacent columns of elongated holes are staggered. Accordingly, the structural strength of the support element 10 can be improved.

In an embodiment, referring to FIG. 6, two adjacent columns of elongated holes are arranged side by side. Accordingly, the aesthetic appeal of the support element 10 is improved while the processing efficiency is improved.

The liquid guiding holes 10b are symmetrically distributed in the body 11, and a symmetric structure of the liquid guiding holes 10b can reduce laser processing costs. Additionally, the liquid guiding holes 10b are symmetrically distributed in the body 11, thereby allowing the aerosol-forming material within the liquid guiding channel 10a to be evenly guided to the liquid guiding assembly 20 via the liquid guiding holes 10b, and then improving the atomization effect. Exemplarily, in an embodiment, the liquid guiding holes 10b are symmetrically distributed along the axial direction of the body 11. In some other embodiments, the liquid guiding holes 10b are symmetrically distributed along the circumferential direction of the body 11. In yet other embodiments, the liquid guiding holes 10b are symmetrically distributed along the central axis of the body 11.

In an embodiment, referring to FIG. 1 to FIG. 3, the length of the body 11 along the axial direction is greater than the length of the liquid guiding assembly 20 along the axial direction. In other words, a part of the body 11 extending out of the liquid guiding assembly 20 along the axial direction may be arranged within the liquid storage cavity 1000a. Therefore, the thermal conduction layer 21 may conduct the heat to the support element 10 more quickly, and then a part of the support element 10 extending into the liquid storage cavity 1000a conducts the heat to the aerosol-forming material within the liquid storage cavity 1000a more quickly. After the aerosol-forming material heats up, the viscosity is reduced, thereby improving the fluidity of the aerosol-forming material nearby the support element 10 within the liquid storage cavity 1000a. Ventilation is improved, and meanwhile the aerosol-forming material is facilitated to be guided to the heating element 30 via the liquid guiding assembly 20, thereby improving the liquid guiding effect and the atomization effect of the heating assembly 100.

In an embodiment, referring to FIG. 1 and FIG. 2, the length of the thermal conduction layer 21 along the axial direction is greater than the length of the isolation layer 22 along the axial direction. That is, the length of the thermal conduction layer 21 along the axial direction of the support element 10 is greater than the length of the isolation layer 22 along the axial direction of the support element 10. Therefore, the contact area between the thermal conduction layer 21 and both the support element 10 and the aerosol-forming material can be increased, thereby further conducting more heat to the support element 10 more quickly, then allowing the support element 10 to conduct the heat to the aerosol-forming material nearby the support element 10 more quickly, reducing the fluidity of the aerosol-forming material nearby the thermal conduction layer 21, and then improving the liquid guiding effect and ventilation of the liquid guiding assembly 20.

In an embodiment, referring to FIG. 5, the inner diameter D of the body 11 ranges from 0.3 mm to 3 mm, and in other words, the diameter of the liquid guiding channel 10a ranges from 0.3 mm to 3 mm. If the inner diameter of the body 11 is too small, that is, the diameter of the liquid guiding channel 10a is too small, the flow of the aerosol-forming material within the liquid guiding channel 10a may be influenced, reducing the liquid supply capacity of the heating assembly 100, leading to decrease in the atomization efficiency of the atomizer 1000. If the inner diameter of the body 11 is too large, the size of the heating assembly 100 becomes large, which on one hand, occupies the internal space of the atomizer 1000, and on the other hand, leads to a large size of the atomizer 1000, compromising the product aesthetics. By limiting the inner diameter of the body 11 to range from 0.3 mm to 3 mm, the aesthetic appeal of the atomizer 1000 is improved while the liquid supply capacity of the heating assembly 100 is ensured. It should be understood that the inner diameter of the body 11 is selected according to actual needs. For example, the inner diameter of the body 11 is determined according to the size of a specific vaping cartridge.

In an embodiment, referring to FIG. 4, the axial length L1 of the body 11 ranges from 3 mm to 30 mm. It should be understood that the longer the axial length of the body 11, the longer the corresponding liquid guiding assembly 20 will be, the larger the area covered by the heating element 30 will be, and the greater the liquid supply area of the liquid guiding holes 10b will be. Accordingly, if the axial length of the body 11 is too short, the liquid supply capacity and the atomization efficiency of the heating assembly 100 are reduced. If the axial length of the body 11 is too large, the size of the heating assembly 100 becomes large, which on one hand, occupies the internal space of the atomizer 1000, and on the other hand, leads to a large size of the atomizer 1000, compromising the product aesthetics. By limiting the axial length of the body 11 to range from 3 mm to 30 mm, the aesthetic appeal of the atomizer 1000 is improved while the liquid supply capacity and the atomization efficiency of the heating assembly 100 are ensured. It should be understood that the axial length of the body 11 is selected according to actual needs. For example, the axial length of the body 11 is determined according to the size of the specific vaping cartridge.

In an embodiment, referring to FIG. 5, the wall thickness L2 of the body 11 ranges from 0.05 mm to 0.2 mm. It should be understood that under the condition of ensuring strength and safety, the wall thickness of the body 11 can be reduced as much as possible, thereby reducing the mass of the body 11, reducing thermal capacity consumption of the body 11, and improving the heating efficiency of the heating assembly 100. Additionally, with the same outer diameter, the inner diameter of the body 11 may be larger, and macroscopic flow resistance in the liquid guiding channel 10a is smaller. Additionally, by reducing the wall thickness of the body 11, a path for the aerosol-forming material to flow from the liquid guiding channel 10a to the liquid guiding assembly 20 is shortened, thereby further reducing the flow resistance of the aerosol-forming material, and then improving the liquid supply capacity and the atomization effect of the heating assembly 100.

In an embodiment, referring to FIG. 4, the width L3 of the elongated holes ranges from 0.3 mm to 0.8 mm. It should be understood that if the width of the elongated holes is too small, the flow of the aerosol-forming material is not facilitated, reducing the liquid supply capacity of the heating assembly 100 and lowering the atomization efficiency of the atomizer 1000. If the width of the elongated holes is too large, the structural strength of the support element 10 is reduced, and consequently, the service life of the heating assembly 100 is shortened. Meanwhile, the too large width of the elongated holes may lead to liquid leakage. By limiting the width of the elongated holes to range from 0.3 mm to 0.8 mm, the service life of the heating assembly 100 can be prolonged while the atomization efficiency of the atomizer 1000 is ensured.

In an embodiment, please continue to refer to FIG. 4, the length L4 of the elongated holes ranges from 1 mm to 3 mm. It should be understood that if the length of the elongated holes is too small, the flow of the aerosol-forming material is not facilitated, reducing the liquid supply capacity of the heating assembly 100 and lowering the atomization efficiency of the atomizer 1000. If the length of the elongated holes is too large, the structural strength of the support element 10 is reduced, and consequently, the service life of the heating assembly 100 is shortened. Meanwhile, the too large width of the elongated holes may lead to liquid leakage. By limiting the length of the elongated holes to range from 1 mm to 3 mm, the service life of the heating assembly 100 can be prolonged while the atomization efficiency of the atomizer 1000 is ensured.

In an embodiment, please continue to refer to FIG. 4, a spacing L5 between the two adjacent rows of elongated holes distributed along the axial direction of the body 11 ranges from 0.6 mm to 1.2 mm. It should be noted that the spacing between the two adjacent rows of elongated holes distributed along the axial direction of the body 11 resembles micro-ribs between the two adjacent rows of elongated holes. That is, the two adjacent rows of elongated holes are connected through the micro-ribs to ensure the strength of the support element 10. If the spacing between the two adjacent rows of elongated holes is too small, the structural strength of the support element 10 is reduced, and consequently, the service life of the heating assembly 100 is shortened. If the spacing between the two adjacent rows of elongated holes is too large, the liquid supply area of the support element 10 may be reduced, and consequently, the liquid supply capacity and the atomization efficiency of the heating assembly 100 are reduced. By limiting the spacing between the two adjacent rows of elongated holes distributed along the axial direction of the body 11 to range from 0.6 mm to 1.2 mm, the service life of the heating assembly 100 can be prolonged while the liquid supply capacity and the atomization efficiency of the atomizer 1000 are ensured.

In an embodiment, please continue to refer to FIG. 4, the spacing L6 between the elongated hole closest to an end portion of the body 11 and the end portion of the body 11 ranges from 1 mm to 3 mm. It should be noted that the elongated hole closest to the end portion of the body 11 is an edge hole in each of the two ends of the body 11. If the spacing between the edge hole and the end portion of the body 11 is too large, the liquid supply area of the support element 10 may be reduced, and consequently, the liquid supply capacity and the atomization efficiency of the heating assembly 100 are reduced. If the spacing between the edge hole and the end portion of the body 11 is too small, the structural strength of the support element 10 is reduced, and consequently, the service life of the heating assembly 100 is shortened. By limiting the spacing between the elongated hole closest to the end portion of the body 11 and the end portion of the body 11 to range from 1 mm to 3 mm, the service life of the heating assembly 100 can be prolonged while the atomization efficiency of the atomizer 1000 is ensured.

In a specific embodiment, the axial length L1 of the body 11 is 8 mm, the length L4 of the elongated holes is 1.8 mm, the width L3 of the elongated holes is 0.5 mm, and the spacing L5 between the two adjacent rows of elongated holes distributed along the axial direction of the body 11 is 0.8 mm.

In some other embodiments, the equivalent hole diameter of the liquid guiding holes 10b ranges from 0.01 mm to 3 mm, thereby avoiding the situation that the flow of the aerosol-forming material is influenced by the too small hole diameter, reducing the liquid supply capacity of the heating assembly 100; the structural strength of the support element 10 is reduced due to the too large hole diameter, shortening the service life of the heating assembly 100; and meanwhile the too large hole diameter may cause liquid leakage.

In an embodiment, referring to FIG. 1 and FIG. 2, the length of the thermal conduction layer 21 along the axial direction of the support element 10 is greater than the length of the isolation layer 22 along the axial direction. Therefore, the contact area between the thermal conduction layer 21 and both the support element 10 and the aerosol-forming material can be increased, thereby further transferring more heat to the aerosol-forming material, to reduce the fluidity of the aerosol-forming material nearby the thermal conduction layer 21, and then improve the liquid guiding effect and ventilation of the liquid guiding assembly 20.

In the descriptions of this specification, descriptions using reference terms “an embodiment”, “some embodiments”, “some other embodiment”, “yet other embodiments”, or “exemplarily” mean that specific features, structures, materials, or characteristics described with reference to the embodiments or examples are included in at least one embodiment or example of the embodiments of this application. In this application, exemplary expressions of the above terms do not necessarily refer to the same embodiment or example. Besides, the specific features, the structures, the materials, or the characteristics that are described may be combined in proper manners in any one or more embodiments or examples. Additionally, without mutual contradiction, those skilled in the art may combine different embodiments or examples described in this application, as well as features of the different embodiments or examples.

The above contents are merely preferred embodiments of this application and are not used for limiting this application, and this application may be variously modified and changed for those skilled in the art. Any modification, equivalent substitution, improvement, etc. made within the spirit and principle of this application shall fall within the scope of protection of this application.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A liquid guiding assembly for an electronic atomization device, comprising:
at least one thermal conduction layer and at least one isolation layer that are stacked,
wherein the at least one isolation layer makes contact with a heating element of the electronic atomization device, the at least one isolation layer isolating the thermal conduction layer from the heating element, and
wherein the at least one isolation layer is configured to transfer at least some heat generated by the heating element to the at least one thermal conduction layer.

2. The liquid guiding assembly of claim 1, wherein the at least one isolation layer is sleeved over a periphery of the at least one thermal conduction layer.

3. The liquid guiding assembly of claim 2, wherein a length of the at least one thermal conduction layer along an axial direction is greater than a length of the at least one isolation layer along the axial direction.

4. The liquid guiding assembly of claim 1, wherein the at least one thermal conduction layer comprises a metal mesh layer.

5. The liquid guiding assembly of claim 4, wherein the at least one thermal conduction layer comprises a copper alloy.

6. The liquid guiding assembly of claim 1, wherein the at least one isolation layer comprises a cotton layer.

7. The liquid guiding assembly of claim 1, wherein a porosity of the at least one thermal conduction layer ranges from 0.45 to 0.99, and/or
wherein a permeability of the at least one thermal conduction layer ranges from 1×10⁻¹¹ m² to 1×10⁻⁹ m².

8. The liquid guiding assembly of claim 1, wherein a porosity of the at least one isolation layer ranges from 0.45 to 0.99, and/or
wherein a permeability of the at least one isolation layer ranges from 1×10⁻¹¹ m² to 1×10⁻⁹ m².

9. The liquid guiding assembly of claim 1, wherein the at least one thermal conduction layer comprises a plurality of thermal conduction layers,
wherein the at least one isolation layer comprises a plurality of isolation layers, and
wherein the plurality of thermal conduction layers and the plurality of isolation layers are alternately arranged.

10. A heating assembly, comprising:
a heating element; and
the liquid guiding assembly of claim 1,
wherein the heating element is arranged on the at least one isolation layer.

11. The heating assembly of claim 10, further comprises:
a support element comprising a body, a liquid guiding channel, and liquid guiding holes, the liquid guiding channel penetrating through two ends of the body along an axial direction of the body, the liquid guiding holes penetrating through a side wall of the liquid guiding channel along a radial direction of the body,
wherein the at least one thermal conduction layer is sleeved over a periphery of the support element, the at least one isolation layer is sleeved over a periphery of the at least one thermal conduction layer, and an aerosol-forming material within the liquid guiding channel is guidable to the liquid guiding assembly via the liquid guiding holes.

12. The heating assembly of claim 11, wherein the support element comprises a metal element.

13. The heating assembly of claim 11, wherein a length of the body along an axial direction is greater than a length of the liquid guiding assembly along the axial direction of the body.

14. The heating assembly of claim 11, wherein an equivalent hole diameter of the liquid guiding holes ranges from 0.01 mm to 3 mm.

15. The heating assembly of claim 11, wherein an inner diameter of the body ranges from 0.3 mm to 3 mm, and/or
wherein an axial length of the body ranges from 3 mm to 30 mm, and/or
wherein a wall thickness of the body ranges from 0.05 mm to 0.2 mm.

16. An atomizer, comprising:
a liquid storage cavity; and
the heating assembly of claim 10,
wherein the liquid storage cavity is configured to store an aerosol-forming material, and
wherein the aerosol-forming material within the liquid storage cavity is guidable to the heating element via the liquid guiding assembly.

17. An electronic atomization device, comprising:
a power supply assembly; and
the atomizer of claim 16,
wherein the power supply assembly is electrically connected with the heating assembly.
